(19) United States
(12) Patent Application Publication
(10) Pub. No.: US 2010/0262031 A1
(43) Pub. Date: Oct. 14, 2010

(54) Method and system for respiratory phase classification using explicit labeling with label verification

(76) Inventors: Yongji Fu, Aloha, OR (US); Nhedti Colquitt, Aloha, OR (US); Deepak Ayyagari, Vancouver, WA (US); Jingping Xu, Vancouver, WA (US)

Correspondence Address: SHARP LABORATORIES OF AMERICA, INC., 1320 PEARL ST., SUITE 228, BOULDER, CO, 80302, US

(21) Appl. No.: 12/386,072

(22) Filed: Apr. 14, 2009

(51) Int. Cl. A61B 5/08

(52) U.S. Cl. 600/529

(57) ABSTRACT

A method and system classify respiratory phases in a single channel acoustic signal as inspiratory and expiratory using explicit labeling with label verification. In the method and system, a subject explicitly indicates through a user input the start of a respiratory cycle (i.e. start of inspiration). The phase indication is applied to provisionally label several consecutive phases of a single channel acoustic signal as inspiratory and expiratory. A provisional phase rule set is then generated based on characteristic differences between the inspiratory and expiratory phases. The phase indication, provisional labeling and provisional rule set generation steps are then repeated. The two generated provisional rule sets are then compared for a match to verify the accuracy of the subject's phase indications and the ability to automatically recover phase in the event of signal loss.

### BACKGROUND OF THE INVENTION

The present invention relates to respiratory monitoring and, more particularly, to a method and system for classifying respiratory phases in a single channel acoustic signal as inspiratory and expiratory using explicit labeling with label verification, and for automatically recovering phase after signal interruption. Without extra channels, the present invention addresses the difficulty in distinguishing between the inspiratory and expiratory phases that arises from the lack of a universal signal characteristic to differentiate these phases for all people. Moreover, the present invention provides for automatic recovery of phase after a loss of phase tracking due to reasons such as loss of signal or noisy signal.

Respiration in humans is typically characterized by two phases: inspiration, or the intake of air into the lungs, and expiration, or the expelling of air from the lungs. Data that characterize respiratory phases is very important in individual respiratory health determinations and the study of pulmonary diseases. For example, a low fractional inspiratory time (i.e. inspiratory phase time divided by respiratory cycle time) or a low inspiratory to expiratory time ratio (i.e. inspiratory phase time divided by expiratory phase time, also known as I:E ratio) may reflect a prolonged expiratory phase that is indicative of obstruction of the airways. A high fractional inspiratory time or I:E ratio may be used to inform as to the present status of a monitored subject, for example, that the subject is snoring or speaking. The trend in fractional inspiratory time and I:E ratio may also be instructive in some applications.

One method for obtaining respiratory phase data is the lung sound method, sometimes called auscultation. The lung sound method has become increasingly popular due in part to the low cost and ready availability of lung sound detection systems. In the lung sound method, one or more body mounted respiratory sound transducers records sounds from which respiratory phase data are determined. Tracheal sounds, typically heard over the supreasternal notch or at the lateral neck near the pharynx, are often chosen for respiratory sound detection because the sounds have a high signal-to-noise ratio and a high sensitivity to variation in flow that enable accurate determination of respiratory phase starting points.

One problem with known implementations of the lung sound method is the inability to distinguish inspiratory and expiratory phases of a respiration cycle from a single sound transducer (i.e. single channel sound signal). There is no universal signal characteristic to differentiate the inspiratory and expiratory phases for all people. For example, the difference in amplitude between inspiratory and expiratory tracheal sounds varies greatly among subjects. For many people, inspiratory sounds are louder while for others there is not much difference and for still others expiratory sounds are louder. Therefore, distinguishing between the inspiratory and expiratory phases of a respiratory cycle can be difficult using tracheal sounds alone. One way to address this shortcoming of the lung sound method is to install additional sound transducers on parts of the subject's body, such as on the subject's chest and/or back, that generate lung sounds from which the inspiratory and expiratory phases can be better distinguished. However, reliance on additional sound transducers (i.e. multi-channel sound signal) can add to subject discomfort as well as system complexity and computational overhead.

Another problem with known implementations of the lung sound method is phase recovery after signal interruption. A method for identifying inspiratory and expiratory phases may lose track of phase for any number of reasons. One reason may be signal loss due to unreliable network connectivity. Another reason may be a noisy signal induced by, for example, the surrounding environment, the subject's speech or the subject's motion. Requiring the subject to manually intervene to recover phase every time there is a loss of phase tracking is burdensome and can be a cause of frustration.

### SUMMARY OF THE INVENTION

The present invention, in a basic feature, provides a method and system for classifying respiratory phases in a single channel acoustic signal as inspiratory and expiratory using explicit labeling with label verification. In the method and system, a subject explicitly indicates through a user input the start of a respiratory cycle (i.e. start of inspiration). The phase indication is applied to provisionally label several consecutive phases of a single channel acoustic signal as inspiratory and expiratory. A provisional phase rule set is then generated based on characteristic differences between the inspiratory and expiratory phases. The phase indication, provisional labeling and provisional rule set generation steps are then repeated. The two generated provisional rule sets are then compared for a match to verify the accuracy of the subject's phase indications and the ability to automatically recover phase in the event of signal loss.

In one aspect of the invention, a system for classifying respiratory phases comprises a data processor, a respiratory sound transducer and a user interface, wherein the data processor receives a respiratory signal from the respiratory sound transducer and an first respiratory phase indication from the user interface and labels a first multiple of respiratory phases in the respiratory signal as inspiratory and a first multiple of respiratory phases in the respiratory signal as expiratory based on the first respiratory phase indication.

In some embodiments, the data processor generates a first phase rule set based on a comparison of characteristics of the first multiple of respiratory phases labeled as inspiratory with the first multiple of respiratory phases labeled as expiratory.

In some embodiments, the first phase rule set comprises rules indicative of differences between the first multiple of respiratory phases labeled as inspiratory and the first multiple of respiratory phases labeled as expiratory.

In some embodiments, the first phase rule set comprises a rule indicative of a difference in one or more of phase duration, maximum amplitude, slope from start of phase to maximum amplitude, slope from maximum amplitude to end of phase, phase width or phase surface area under envelope.

In some embodiments, the data processor receives a second respiratory phase indication from the user interface and labels a second multiple of respiratory phases in the respiratory signal as inspiratory and a second multiple of respiratory phases in the respiratory signal as expiratory based on the second respiratory phase indication, wherein the data processor generates a second phase rule set based on a comparison of characteristics of the second multiple of respiratory phases labeled as inspiratory and the second multiple of respiratory phases labeled as expiratory, and wherein the data processor compares the first and second phase rule sets for a match and generates a permanent phase rule set in response to a match.

In some embodiments, the data processor automatically labels a third multiple of respiratory phases in the respiratory signal as inspiratory and a third multiple of respiratory phases in the respiratory signal as expiratory based on the permanent phase rule set.

In some embodiments, the data processor performs the automatic labeling after recovery from a temporary interruption of the respiratory signal.

In another aspect of the invention, a method for classifying respiratory phases comprises the steps of receiving a respiratory signal, receiving a first respiratory phase indication based on a first user input and labeling a first multiple of respiratory phases in the respiratory signal as inspiratory and a first multiple of respiratory phases in the respiratory signal as expiratory based on the first respiratory phase indication.

In some embodiments, the method further comprises the step of generating a first phase rule set based on a comparison of characteristics of the first multiple of respiratory phases labeled as inspiratory with the first multiple of respiratory phases labeled as expiratory.

In some embodiments, the first phase rule set comprises rules indicative of differences between the first multiple of respiratory phases labeled as inspiratory and the first multiple of respiratory phases labeled as expiratory.

In some embodiments, the first phase rule set comprises a rule indicative of a difference in phase duration.

In some embodiments, the first phase rule set comprises a rule indicative of a difference in maximum amplitude.

In some embodiments, the first phase rule set comprises a rule indicative of a difference in slope from start of phase to maximum amplitude.

In some embodiments, the first phase rule set comprises a rule indicative of a difference in slope from maximum amplitude to end of phase.

In some embodiments, the first phase rule set comprises a rule indicative of a difference in phase width.

In some embodiments, the first phase rule set comprises a rule indicative of a difference in phase surface area under envelope.

In some embodiments, the method further comprises the steps of receiving a second respiratory phase indication based on a second user input, labeling a second multiple of respiratory phases in the respiratory signal as inspiratory and a second multiple of respiratory phases in the respiratory signal as expiratory based on the second respiratory phase indication, generating a second phase rule set based on a comparison of characteristics of the second multiple of respiratory phases labeled as inspiratory and the second multiple of respiratory phases labeled as expiratory, comparing the first and second phase rule sets for a match and generating a permanent phase rule set in response to a match.

In some embodiments, the method further comprises the step of automatically labeling a third multiple of respiratory phases in respiratory signal as inspiratory and a third multiple of respiratory phases in the respiratory signal as expiratory based on the permanent phase rule set.

In some embodiments, the automatic labeling step is performed after recovery from a loss of phase tracking due to a loss of network connectivity.

In some embodiments, the automatic labeling step is performed after recovery from a loss of phase tracking due to a noisy respiratory signal.

In some embodiments, the method further comprises the step of verifying that the phase in the respiratory signal that is addressed by the first respiratory phase indication and the phase in the respiratory signal that is addressed by the second respiratory phase indication are separated by an odd number of intervening phases.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a system for classifying respiratory phases in some embodiments of the invention.

FIG. 2 shows a sequence of user screens presented to a patient in a system for classifying respiratory phases in some embodiments of the invention.

FIG. 3 shows a method for classifying respiratory phases in some embodiments of the invention.

FIG. 4 shows a method for automatically classifying respiratory phases after recovery from a loss of phase tracking in some embodiments of the invention.

FIG. 5 shows an exemplary respiratory signal over several respiratory phases with phase durations, maximum amplitudes and slopes from start of phase to maximum amplitude delineated.

FIG. 6 shows an exemplary respiratory signal over several respiratory phases with slopes from maximum amplitude to end of phase delineated.

FIG. 7 shows an exemplary respiratory signal over several respiratory cycles with phase envelope widths delineated.

FIG. 8 shows an exemplary respiratory signal over several respiratory cycles with a phase surface area under envelope delineated.

### DETAILED DESCRIPTION OF A PREFERRED EMBODIMENT

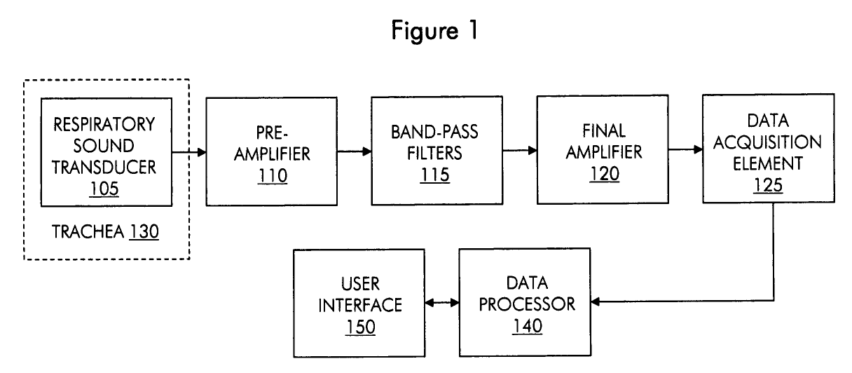

FIG. 1 shows a system for classifying respiratory phases in some embodiments of the invention. The system includes a respiratory sound transducer 105 positioned at the trachea 130 of a human subject being monitored, although in other embodiments transducer 105 may be positioned at the subject's chest or back. Transducer 105 is communicatively coupled in series with a pre-amplifier 110, bond-pass filters 115, a final amplifier 120 and a data acquisition element 125. Data acquisition element 125 transmits a respiratory signal detected by transducer 105, as modified by amplifiers 110, 120 and filters 115, to a data processor 140. In some embodiments, the respiratory signal is a continuous single channel respiratory acoustic signal. Data processor 140 is also communicatively coupled with user interface 150 that receives inputs and transmits outputs to the subject.

In some embodiments, elements 105, 110, 115, 120, 125 and 140 reside on an acoustic transducer device that captures a respiratory signal, provides on-board processing, and has a wireless interface that supports communication with a portable electronic device, such as a mobile phone or personal data assistant (PDA) on which user interface 150 is resident.

In other embodiments, elements 105, 110, 115, 120 and 125 reside on an acoustic transducer device that captures a respiratory signal and has a wireless interface that supports communication with a portable electronic device, such as a mobile phone or PDA on which processor 140 and user interface 150 are resident.

In still other embodiments, elements 105, 110, 115, 120 and 125 reside on an acoustic transducer device that captures a respiratory signal and has a wired interface, such as a Universal Serial Bus (USB) interface, that supports communication with a desktop or notebook personal computer on which processor 140 and user interface 150 are resident.

Transducer 105 detects a respiratory signal at trachea 130. Transducer 105 outputs the detected respiratory signal to pre-amplifier 110 as an analog voltage.

Pre-amplifier 110 provides impedance match for the respiratory signal received from transducer 105 and amplifies the respiratory signal to a level appropriate for the filter stage that follows.

Band-pass filters 115 include an analog high-pass filter that applies a cutoff frequency to the respiratory signal received from pre-amplifier 110 to reduce noise that may include, for example, heart sounds, muscle sounds and contact noise. Filters 115 also include a low-pass filter with a cutoff frequency applied to the respiratory signal following the high-pass filter.

Final amplifier 120 amplifies the respiratory signal received from band-pass filters 115.

Data acquisition element 125 performs analog/digital (A/D) conversion on the respiratory signal received from final amplifier 120 and down-samples, if necessary, the respiratory signal in order to reduce the sampled data length. Data acquisition element 125 transmits the resulting respiratory signal to data processor 140 for analysis.

Data processor 140 is a microprocessor having software executable thereon for classifying respiratory phases in the respiratory signal received from data acquisition element 125, including phase labeling, phase rule set generation and phase rule set comparison, and performing respiratory phase-independent and phase-specific analysis on the respiratory signal. In some embodiments, processor 140 generates respiratory phase data, such as fractional inspiratory time and/or I:E ratio, based on the respiratory signal on a continuous basis to enable real-time monitoring of the respiratory health of a patient.

User interface 150 includes an input device, such as one or more of a keyboard, keypad, touch screen or mouse, through which the subject inputs information, such as a phase indication that indicates the start of a respiratory cycle (i.e. start of inspiration). User interface 150 also includes an output device, such as one or more of an liquid crystal display (LCD) screen or light emitting diode (LED) display screen, on which the subject views various user screens, such as user screens providing explicit phase labeling status information and respiratory health status information.

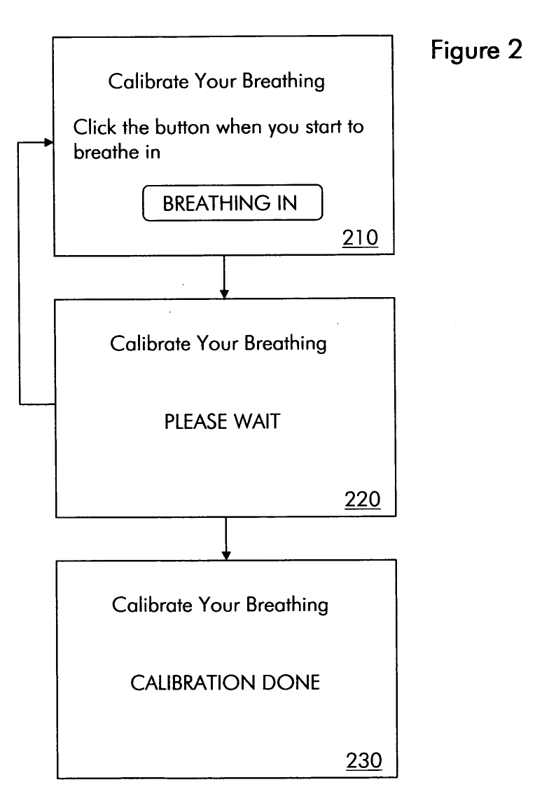

FIG. 2 shows a sequence of user screens presented to a subject in a system for classifying respiratory phases in some embodiments of the invention. A phase indication input instruction screen 210 is first presented. Screen 210 instructs the subject to press the button when the subject starts to breathe-in. The subject presses the button upon breathing-in and the time at which the button is pressed is used by the system to provisionally label as inspiratory the phase of the respiratory signal at the corresponding time. Using that provisional label as a marker, the system provisionally labels additional respiratory phases of the respiratory signal across several consecutive respiratory cycles and generates a first provisional rule set based on characteristic differences between the respiratory phases that have been provisionally labeled as inspiratory and the respiratory phases that have been provisionally labeled as expiratory. While this provisional labeling and provisional rule set generation is ongoing, a wait screen 220 is presented to the subject. Once the provisional rule set generation has been completed, phase indication input instruction screen 210 is presented to the subject in a second instance and the process is repeated. However, after wait screen 220 has been presented and provisional rule set generation completed in a second instance, completion screen 230 is presented.

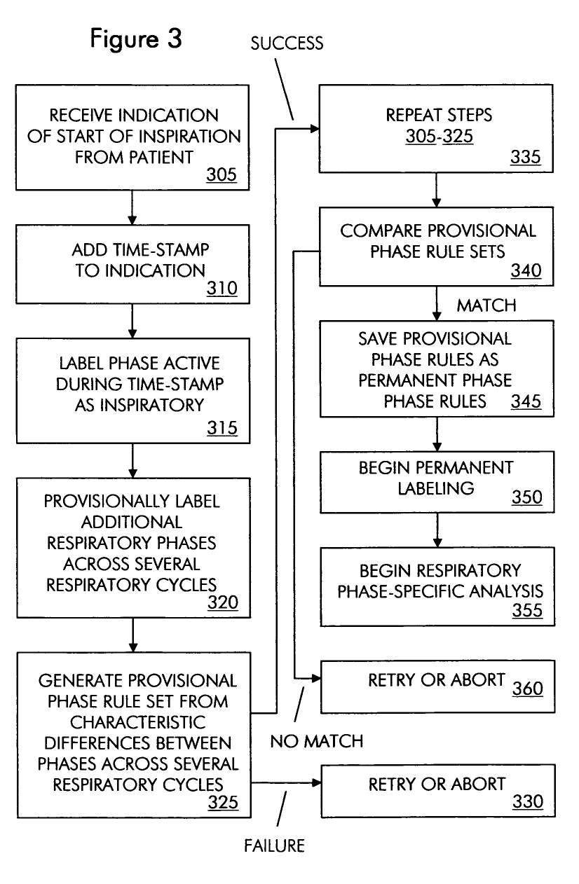

FIG. 3 shows a method for classifying respiratory phases in some embodiments of the invention. Initially, the subject breathes in and inputs on user interface 150 an indication of start of inspiration (305). For example, the subject may press the “BREATHING IN” button on phase indication input instruction screen 210. User interface 150 adds a timestamp to the phase indication (310) and relays the time-stamped phase indication to data processor 140. Data processor 140 analyzes the respiratory signal received from data acquisition element 125 and labels the respiratory phase at the time indicated by the timestamp as inspiratory (315). Data processor 140 proceeds to provisionally label additional respiratory phases across consecutive respiratory cycles as expiratory and inspiratory, in turn, in accordance with the alternating phase (320). In some embodiments, three consecutive respiratory cycles are labeled, resulting in a total of six consecutive respiratory phases (i.e. three inspiratory and three expiratory) being labeled.

Data processor 140 then generates a provisional phase rule set based on an analysis of characteristic differences between the inspiratory and expiratory phases in the six labeled respiratory phases (325). By way of example, characteristic differences that data processor 140 analyzes to generate the provisional rule set may include the following:

(1) Relative phase duration: This check determines whether the inspiratory or expiratory phase is longer. Turning to FIG. 5, phase durations 505, 510 for consecutive phases are delineated. In the check, phase durations of three inspiratory phases are compared with phase durations of three expiratory phases to make the determination.

(2) Relative maximum amplitude: This check determines whether the inspiratory or expiratory phase has a higher maximum amplitude. Referring to FIG. 5, maximum amplitudes 520, 530 for consecutive phases are identified. In the check, maximum amplitudes of three inspiratory phases are compared with maximum amplitudes of three expiratory phases to make the determination.

(3) Relative slope from start of phase to maximum: This check determines whether the inspiratory or expiratory phase has a steeper slope from start of phase to maximum amplitude. Referring to FIG. 5, start-to-peak slopes 515, 525 for consecutive phases are identified. In the check, start-to-peak slopes of three inspiratory phases are compared with start-to-peak slopes of three expiratory phases to make the determination.

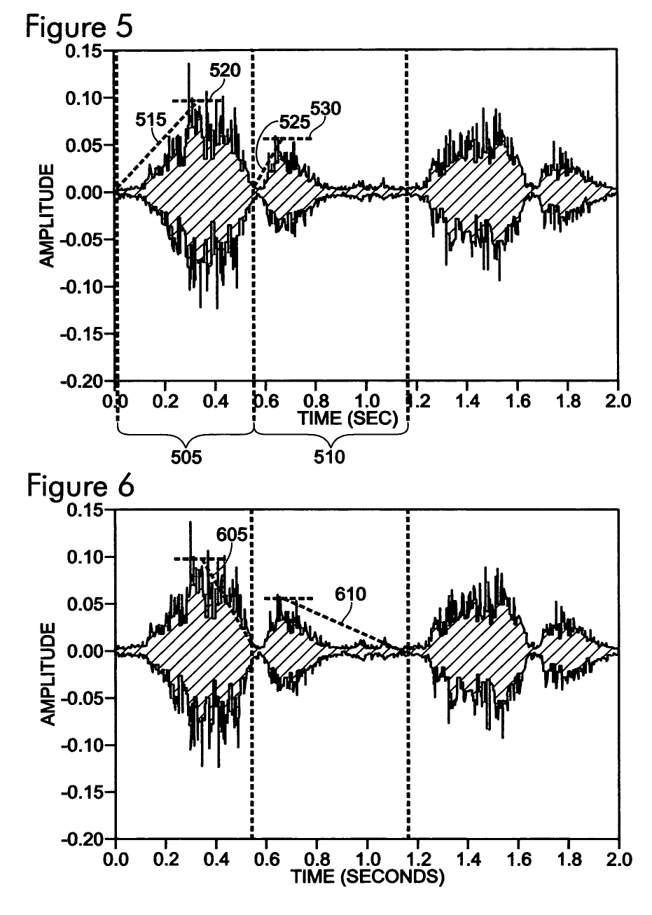

(4) Relative slope from maximum to end of phase: This check determines whether the inspiratory or expiratory phase has a steeper slope from maximum amplitude to end of phase. Referring to FIG. 6, peak-to-end slopes 605, 610 for consecutive phases are identified. In the check, peak-to-end slopes of three inspiratory phases are compared with peak-to-end slopes to three expiratory phases to make the determination.

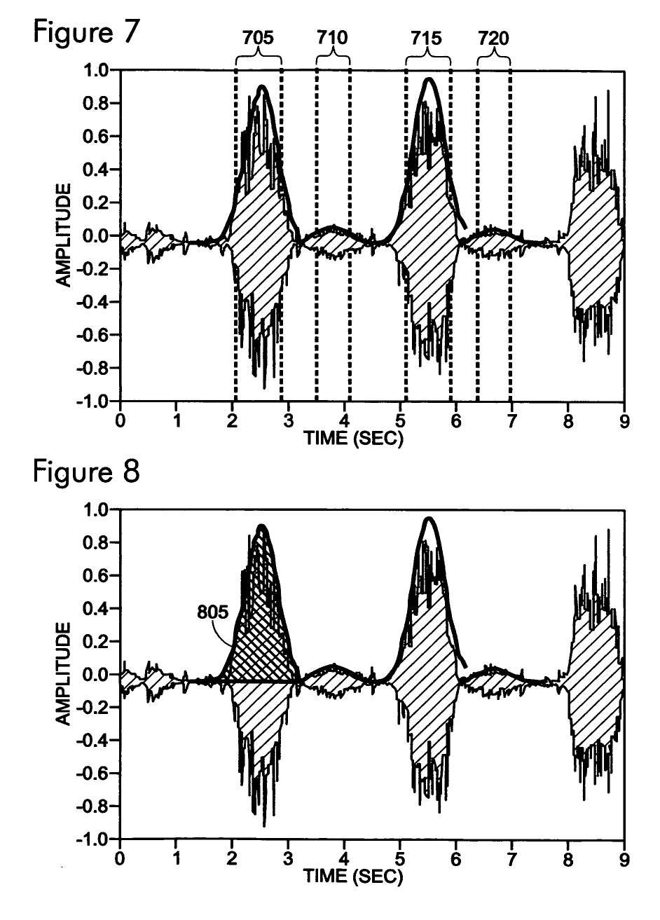

(5) Relative phase width: This check determines whether the inspiratory or expiratory phase is wider. Referring to FIG. 7, phase widths 705, 710, 715, 720 for consecutive phases are delineated. Phase widths 705, 710, 715, 720 are determined by calculating the full width at half maximum of the fitted Gaussian curve of the phase envelope. In the check, phase widths of three inspiratory phases are compared with phase widths of three expiratory phases to make the determination.

(6) Relative phase surface area under envelope: This check determines whether the surface area under envelope of the inspiratory or expiratory phase is larger. Referring to FIG. 8, a phase surface area under envelope 805 is depicted as a region shaded with hatch lines. In the check, the phase surface areas under envelope of three inspiratory phases are compared with phase surface areas under envelope of three expiratory phases to make the determination.

The provisional rule set may include a rule for each characteristic difference expressed in terms of one of four outcomes. One outcome is that the inspiratory phase values for the characteristic are consistently greater than the expiratory phase values. A second outcome is that the expiratory phase values for the characteristic are consistently greater than the inspiratory phase values. A third outcome is that the differences between the inspiratory phase values and the expiratory phase values for the characteristic are insignificant. For example, if the phase duration of each inspiratory phase is 2.01 seconds and the phase duration of each expiratory phase is 1.99 seconds, the third outcome would attach. A fourth outcome is that the differences between the inspiratory phase values and the expiratory phase values for the characteristic, while significant, are inconsistent. For example, if the inspiratory phase has a higher maximum amplitude than the expiratory phase for two out of three respiratory cycles and the expiratory phase has a higher maximum amplitude than the inspiratory phase for the third respiratory cycle, the fourth outcome would attach.

Provisional phase rule sets are expressed in binary codes. For example, for each characteristic, a code of “10” may be used if the inspiratory phase values are consistently greater than the expiratory phase values, “01” may be used if the expiratory phase values are greater than the inspiratory phase values, “00” may be used if the differences between the inspiratory and expiratory phase values are insignificant, and “11” may be used if the differences between the inspiratory and expiratory phase values, while significant, are inconsistent.

If the provisional phase rule set generated in Step 325 indicates no consistent difference for any characteristic, provisional rule set generation is deemed to have failed and a decision is made to either retry (i.e. reperform Steps 305-325) or abort the process (330).

If the provisional phase rule set generated in Step 325 indicates a consistent difference for at least one characteristic, provisional rule set generation is deemed successful and Steps 305-325 are repeated in a second instance (335).

Once two provisional phase rule sets have been successfully generated, the flow advances to Step 340 where the provisional phase rule sets are compared for a match to verify the accuracy of the subject's phase indications and the ability to automatically recover phase in the event of signal loss. A match is found if the binary codes for each characteristic in the provisional phase rule set generated in the first instance match the corresponding binary codes for each characteristic in the provisional phase rule set generated in the second instance. If a match is not found, permanent rule set generation is deemed to have failed and a decision is made to either retry (i.e. reperform Steps 305-325) or abort the process (360). If, however, a match is found, the provisional phase rules are saved as permanent phase rules (345), at which point data processor 140 begins permanent phase labeling based on the verified explicit label (350) and respiratory phase-specific analysis of the respiratory signal (355).

In some embodiments, data processor 140 counts the number of respiratory phases that have passed between the first and second respiratory phase indications and verifies that the phase addressed by the second respiratory phase indication and the phase addressed by the first respiratory phase indication are separated by an odd number of intervening phases before generating a provisional phase rule set based on the second respiratory phase indication. This check insures that both phase indications address the some type of phase (e.g. both inspiratory) and provides an additional safeguard against error in the patient's explicit labeling. If this check fails, a decision is made to either retry or abort the process.

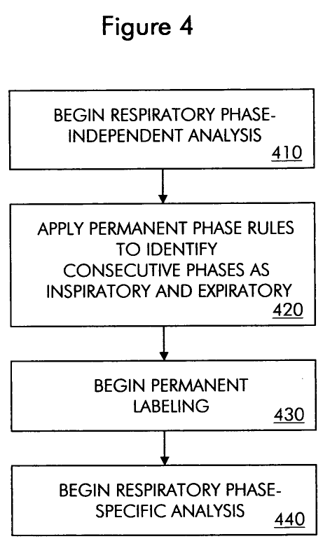

FIG. 4 shows a method for automatically classifying respiratory phases after recovery from a loss of phase tracking in some embodiments of the invention. A loss of phase tracking may occur, for example, if the continuous signal received by data processor 140 from data acquisition element 125 is lost or becomes too noisy. After recovery from such a loss of phase tracking, data processor 140 begins respiratory phase-independent analysis of the respiratory signal received from data acquisition element 125 (410). Data processor 140 then analyzes the respiratory signal in light of the previously saved permanent phase rules to identify consecutive phases as inspiratory and expiratory (420). For example, if the permanent phase rules indicate that the inspiratory phase duration is consistently longer than the expiratory phase duration and that the inspiratory phase has a consistently higher maximum amplitude, data processor 140 uses these characteristic differences to identify a consecutive inspiratory and expiratory phase pair in the respiratory signal and automatically labels them as such. Once consecutive phases have been successfully identified, data processor 140 begins permanent labeling starting with the next phase (430) and begins respiratory phase-specific analysis of the respiratory signal (440).

It will be appreciated by those of ordinary skill in the art that the invention can be embodied in other specific forms without departing from the spirit or essential character hereof. The present description is considered in all respects to be illustrative and not restrictive. The scope of the invention is indicated by the appended claims, and all changes that come with in the meaning and range of equivalents thereof are intended to be embraced therein.

What is claimed is:

1. A system for classifying respiratory phases, comprising:
a data processor;
a respiratory sound transducer; and
a user interface, wherein the data processor receives a respiratory signal from the respiratory sound transducer and an first respiratory phase indication from the user interface and labels a first multiple of respiratory phases in the respiratory signal as inspiratory and a first multiple of respiratory phases in the respiratory signal as expiratory based on the first respiratory phase indication.

2. The system of claim 1, wherein the data processor generates a first phase rule set based on a comparison of characteristics of the first multiple of respiratory phases labeled as inspiratory with the first multiple of respiratory phases labeled as expiratory.

3. The system of claim 2, wherein the first phase rule set comprises rules indicative of differences between the first multiple of respiratory phases labeled as inspiratory and the first multiple of respiratory phases labeled as expiratory.

4. The system of claim 2, wherein the first phase rule set comprises a rule indicative of a difference in one or more of phase duration, maximum amplitude, slope from start of phase to maximum amplitude, slope from maximum amplitude to end of phase, phase width or phase surface area under envelope.

5. The system of claim 2, wherein the data processor receives a second respiratory phase indication from the user interface and labels a second multiple of respiratory phases in the respiratory signal as inspiratory and a second multiple of respiratory phases in the respiratory signal as expirotory based on the second respiratory phase indication, wherein the data processor generates a second phase rule set based on a comparison of characteristics of the second multiple of respiratory phases labeled as inspiratory and the second multiple of respiratory phases labeled as expiratory, and wherein the data processor compares the first and second phase rule sets for a match and generates a permanent phase rule set in response to a match.

6. The system of claim 5, wherein the data processor automatically labels a third multiple of respiratory phases in the respiratory signal as inspiratory and a third multiple of respiratory phases in the respiratory signal as expiratory based on the permanent phase rule set.

7. The system of claim 6, wherein the data processor performs the automatic labeling after recovery from a temporary interruption of the respiratory signal.

8. A method for classifying respiratory phases, comprising the steps of:
receiving a respiratory signal;
receiving a first respiratory phase indication based on a first user input; and
labeling a first multiple of respiratory phases in the respiratory signal as inspiratory and a first multiple of respiratory phases in the respiratory signal as expiratory based on the first respiratory phase indication.

9. The method of claim 8, further comprising the step of generating a first phase rule set based on a comparison of characteristics of the first multiple of respiratory phases labeled as inspiratory with the first multiple of respiratory phases labeled as expiratory.

10. The method of claim 9., wherein the first phase rule set comprises rules indicative of differences between the first multiple of respiratory phases labeled as inspiratory and the first multiple of respiratory phases labeled as expiratory.

11. The method of claim 9,-wherein the first phase rule set comprises a rule indicative of a difference in phase duration.

12. The method of claim 9, wherein the first phase rule set comprises a rule indicative of a difference in maximum amplitude.

13. The method of claim 9, wherein the first phase rule set comprises at least one of a rule indicative of a difference in slope from start of phase to maximum amplitude or a rule indicative of a difference in slope from maximum amplitude to end of phase.

14. The method of claim 9, wherein the first phase rule set comprises a rule indicative of a difference in phase width.

15. The method of claim 9, wherein the first phase rule set comprises a rule indicative of a difference in phase surface area under envelope.

16. The method of claim 9, further comprising the steps of:
receiving a second respiratory phase indication based on a second user input;
labeling a second multiple of respiratory phases in the respiratory signal as inspiratory and a second multiple of respiratory phases in the respiratory signal as expiratory based on the second respiratory phase indication;
generating a second phase rule set based on a comparison of characteristics of the second multiple of respiratory phases labeled as inspiratory and the second multiple of respiratory phases labeled as expiratory;
comparing the first and second phase rule sets for a match; and
generating a permanent phase rule set in response to a match.

17. The method of claim 16, further comprising the step of automatically labeling a third multiple of respiratory phases in respiratory signal as inspiratory and a third multiple of respiratory phases in the respiratory signal as expiratory based on the permanent phase rule set.

18. The method of claim 17, wherein the automatic labeling step is performed after recovery from a loss of phase tracking due to a loss of network connectivity.

19. The method of claim 17, wherein the automatic labeling step is performed after recovery from a loss of phase tracking due to a noisy respiratory signal.

20. The method of claim 16, further comprising the step of verifying that the phase in the respiratory signal that is addressed by the first respiratory phase indication and the phase in the respiratory signal that is addressed by the second respiratory phase indication are separated by an odd number of intervening phases.
